(19) United States
(12) Patent Application Publication
(10) Pub. No.: US 2012/0179127 A1
(43) Pub. Date: Jul. 12, 2012

(54) WOUND CARE ARTICLE COMPRISING A CONVEX INSERT

(76) Inventor: Birgit Riesinger, Ostbevern (DE)

(21) Appl. No.: 13/391,717

(22) PCT Filed: Aug. 23, 2010
(86) PCT No.: PCT/EP10/62215

§ 371 Date: Mar. 26, 2012

(30) Foreign Application Priority Data
Aug. 24, 2009 (DE) 102009038387.5

(51) Int. Cl. A61F 13/45; A61L 15/22

(52) U.S. Cl. 604/369; 604/385.01; 604/367

(57) ABSTRACT

The invention relates to a wound care article having at least one flat layer that contains an absorbent material. Said article is characterised in that it is equipped with at least one other active element that gives the wound care article a convex or pyramidal shape (when viewed from the side).

The invention relates to a wound care article comprising a convex insert according to the characterizing clause of claim 1.

Wound care articles with a wound exudate absorbing body are known from the prior art, for example from DE10059439 of the applicant of the present application. In the process the latter exhibits an essentially flat material segment made of absorption material consisting of an absorbent fleece with superabsorbent particles distributed therein.

These wound care articles have proven themselves extraordinarily well in practice, since they are able to absorb exudates from great depths and thus can considerably improve wound healing in the case of chronic and/or edema induced wounds.

However, these wound care articles are only suitable without further ado for relatively flat wounds. Deep wounds on the other hand require a wound filling material. For this purpose the wound must thus first be filled with a suitable filler, and subsequently the cited wound care article is applied. In many cases this requires an inconvenient handling, impairs among other things the absorptive capacity of the wound care article and involves sepsis issues. In addition the fact must be considered that a bandage produced in such a way comes apart easily and contents, for example particles from superabsorbent polymers, can scatter, which is not desirable.

The present invention addresses the problem of providing a wound care article which is in particular suitable for the treatment of deep wounds.

This problem is solved by the features of the submitted principal claim.

Accordingly a wound care article exhibiting at least one flat layer containing an absorbent material is provided. The wound care article exhibits at least one further active element which gives the wound care article a convex or pyramidal shape when viewed from the side.

The term “convex or pyramidal shape” implies that the wound care article is not flat on the side facing the wound, but rather is arranged in such a way that it can fill deep wounds and comes into contact with the base of the wound over wide areas. In this way deep edema and exudate accumulations are also covered, and the wound healing process is promoted.

In addition, the inventive wound care article makes other wound fillers, as they are used in the state of the art (see above), expendable.

Especially preferably provision can be made that the relevant active elements are equipped as deposits and contain specific active ingredients (for example a nutritive active ingredient, a disinfectant or decontaminating active ingredient and/or a protease inhibiting active ingredient and/or active ingredient complex) or superabsorbent polymers.

Through the cited convex shape these active ingredients or superabsorbent polymers are placed quite close to the active site—in contrast to the above described wound fillers, which as a rule are passive, that is, not equipped with active agents.

This is particularly advantageous, since superabsorbent polymers can bind proteins and bacteria, as e.g. shown in DE102007054127 from the applicant of the present invention. Therefore the closer they are brought to the wound, the more advantageous the affect this property has on the healing of wounds.

Preferably in the process provision is made that the aforementioned at least one active element is a pyramidal, cushion-like, prismatic or hemispherical active element made of a soft and/or elastic material.

Especially preferably provision is made that said soft and/or elastic material is selected from the group containing microspheres, foam, silicone, hydrocolloid material, mull, gauze, fluff and/or dental impression material.

Microspheres are for example known from travel pillows which obtain a plastic deformability from this fill. The aforementioned spheres consist preferably of polystyrene; especially preferably they can be mixed with superabsorbent polymers.

The term “foam” comprises both open-cell and also closed-cell foams made of both natural materials (e.g. starch, cellulose) and synthetic materials (e.g. polyurethane). Foam can in particular be provided with superabsorbent polymers.

The term “hydrocolloid material” refers to plastic materials that are deformable and modellable, as used for example in the field of stoma care. In this connection it is for example a matter of mixtures containing butyl ester of PVM/MA, copolymer, ethanol, gelatins, pectin, plyacrylamide, polysorbate, silica and/or n-butyl-alcohol.

The term “dental impression material” likewise refers to plastic materials that are deformable and modellable, as used for example in dental medicine.

The aforementioned material can if necessary contain boreholes or stamping in order to facilitate the passage of fluid. In principle it is also advantageous if the material is permeable to fluid and/or water vapor.

Further provision is preferably made that in the case of the at least one active element it is a matter of a multi-layer arrangement of flat, individually wrapped layers varying in size.

Said individually wrapped flat layers containing an absorbent material in the process are present in cascaded arrangement, i.e. the smallest layer is arranged on top, while the largest layer is arranged at the bottom.

In the process, provision can for example be made that the smallest layer is 4≦cm² (for example a rectangular size of 1.3×2 cm).

In this connection it is important that the said flat layers are each individually wrapped. In this way swollen superabsorbent particles are prevented from escaping from the individual layers after exudate absorption and scattering in the wound care article, which would lead to a loss of the convex or pyramidal shape.

The flat layer containing an absorbent material has in particular the job of absorbing wound exudate and if necessary actively suctioning said wound exudate. For this purpose the layer must comprise a material that exhibits a swelling capacity or if necessary even deploys osmotic forces.

Especially preferably provision is made in this connection that at least one of the said layers exhibits a segment made of fleece, preferably one made of airlaid.

The term “fleece” denotes a textile flexible sheet material made of individual fibers or filaments which, in contrast to woven fabrics and knitted fabrics is not manufactured from yarn. Fleeces retain their structural integrity as a rule by means of adhesion of the individual fibers to one another. They are also referred to as “non-wovens” and e.g. are produced by milling the fibers. The term “airlaid” denotes a special non-woven fabric made of cellulose and polyolefin fibers in which if necessary superabsorbent polymers are embedded.

Especially preferably provision is made that the wound care article contains superabsorbent polymers at least in sections.

Superabsorbent polymers (SAP) are synthetic materials which are able to absorb many times their own weight—up to 1000 times—in fluids. From a chemical standpoint, it is a matter of a copolymer made of acrylic acid (propenoic acid, C₃H₄O₂) and sodium acrylate (sodium salt of the acrylic acid NaC₃H₃O₂), wherein the proportion of the two monomers to one another can vary. In addition a so-called core cross linker, CXL is added to the monomer solution which bonds the formed long-chain polymer molecules to one another in places by chemical bridges (cross-links them). Through these bridges the polymer becomes water insoluble. In the case of the penetration of water or aqueous salt solutions into the polymer particle it swells up and tautens on the molecular plane of this network so that the water can no longer escape without help.

The superabsorbent polymers can be present in the inventive wound care article in the form of granulates, powders, fills, pellets, foams, in the form of fibers, of a knitted fiber fabric, fiber non-woven and/or of a fiber lap.

The wound care article can preferably exhibit an essentially flat material segment made of absorption material consisting of an absorbent fleece with superabsorbent particles distributed therein. These can be present in the form of granulates, powders, fills, pellets, foams, in the form of fibers, of a knitted fiber fabric, fiber non-woven and/or of a fiber lap.

In the process the wound exudate absorbing body exhibits at least one material which is selected from the group containing a mat, in particular made of an airlaid made of said yarns or fibers made of superabsorbent polymers with incorporated superabsorbent polymers, and/or a loose fill made of superabsorbent polymers. Said airlaid mat can preferably exhibit an essentially flat material segment made of absorption material which e.g. consists of an absorbent fleece made of the named fibers with superabsorbent polymers distributed therein.

This wound exudate absorbing body can correspond to the absorbing insert which is contained in a wound dressing of the applicant of the present invention, as she discloses for example in WO03094813, WO200705199 and WO0152780 and is marketed under the trade name of “sorbion sachet.” The disclosure content of the named publications is attached in its entirety to the disclosure content of this publication.

The wound exudate absorption body can in another embodiment likewise form a core which consists of if applicable flocculent—fibers or yarns made of superabsorbent polymers as well as superabsorbent polymers in granulate form, wherein the granulates are adhered or welded on to the fibers or the yarns at several elevations, and the granulates are distributed over more than 50% of the total overall height of at least one section of the core, wherein blended regions of granulate and fibers are present. The percentage by weight of the superabsorbent polymers can in the process preferably range between 10-25 percent by weight. Similar constructions are known from conventional incontinence materials and are known for their cushioning properties in the case of hygienic dressings. A sleeve can be arranged around the aforementioned core, which is arranged overlapping in regions and which e.g. covers a bonded seam or is part of the same.

Likewise, a section of a hydrophobic and/or water-repellent or waterproof material can be provided within the sleeve, said section acting as drench or laundry protection.

The wound exudate absorption body can in another embodiment likewise contain at least one flat layer exhibiting fibers or yarns to which superabsorbent polymers are adhered in granulate form. As a result of this in a preferable embodiment a structure of the body arises which exhibits at least three layers, wherein two covering layers enclose one layer exhibiting superabsorbent polymers.

In the process on the flat side there are no blends of fibers and superabsorbent polymers; but rather only fixed adjacent areas of both materials. The several layers provided if applicable can in the process in a preferable embodiment also be compressed with each other by rolling, pressing, calendering or similar methods. Moreover the body can exhibit repeating patterns or grains, such as e.g. a check pattern, an embossed pattern or the like.

Further provision is preferably made that the wound care article additionally exhibits a liquid permeable sleeve.

The latter encloses the material segment, forms a barrier against solid excretions and facilitates the passage of other leaked substances to the material segment made of absorption material within the sleeve. The sleeve is preferably closed at least partially by a seam.

Especially preferably the material segment exhibits in top view on its flat side an area (F1) which in its non-wetted state is 3% to 75% smaller than the area (F2) of the sleeve laid flat and is freely moveable or is fixed in the sleeve, wherein the sleeve exhibits pores over the entire surface which however are smaller than the non-wetted superabsorbent polymers.

In this way it is guaranteed that in the case of fluid absorption the material segment can increase in volume and is not restricted by the sleeve.

Especially preferably provision is made that the sleeve in top view on its flat side exhibits a rotary protrusion protruding beyond the seam and that the absorption body is free from hard, sharp edges and corners.

The pores or meshes of the sleeve are preferably 0.05 mm to 1.0 mm, preferably 0.20 mm to 0.50 mm in size. In addition provision can preferably be made that the pores or meshes are bordered by the thread or fiber segments, which in the section through the sleeve are somewhat arched and point outwards with their arch vertices.

The inside layer of the absorption body preferably exhibits an area-specific weight of at least 300 g/m², wherein the square measure of the portion of the osmotically acting substance uniformly distributed therein amounts to at least 100 g/m².

Especially preferably provision is made that the inner layer consists of more than 40 percent by weight superabsorbent polymers.

The wrapping is preferably formed of woven or fleece-like composed synthetic fibers, such as polypropylene or polyethylene fibers, or cotton, silk or rayon. The wrapping consists preferably of a fabric or a fleece, which exhibits an area-specific weight of at least 20 g/m².

Preferably the sleeve is made out of a hydrophobic material, or the sleeve material is hydrophobically equipped. The hydrophobic properties of the sleeve prevent it from adhering to the surface of the wound and assist the wound exudate in reaching the interior of the sleeve more quickly.

In the process provision can be made that the sleeve exhibits an elastic material at least in sections, thus for example fibers made of Lycra. Also this guarantees that the material segment can increase in volume in the case of fluid absorption and is not restricted by the sleeve.

Wound dressings of the named type are for example disclosed in WO03094813, WO200705199 and WO0152780 of the applicant of the present invention. The disclosure content of the named publications is attached in its entirety to the disclosure content of this publication.

The material of the sleeve can be structured in such a way that the sleeve exhibits a rough interior surface and a smooth exterior surface. Preferably the rough interior surface of the sleeve is formed by funnel-shaped perforations which taper in the direction of the interior surface and end in a free opening edge (“overhang”). This rough interior surface counteracts the displacements of the content of the sleeve, so that a fixation with adhesion points can be dispensed with. Accordingly the smooth exterior surface of the sleeve material can be formed by curved, material sections extending between the perforations. Such a sleeve material can, in contrast to a two-sided plane material, be referred to as “three-dimensional”, and is known e.g. from DE102006017194 of the applicant of the present application, to whose disclosure reference is made here completely.

Especially preferably said three-dimensional sleeve material is laminated on the previously mentioned polypropylene non-woven fabric. Such a design results in improved fluid intake properties.

Likewise provision can be made that the wound care article exhibits a fluid -repellent laundry protection (backsheet).

The present invention will be explained more closely by shown and discussed in the following. In the process it is to be kept in mind that the figures only have describing character and are not intended to restrict the invention in any form whatsoever.

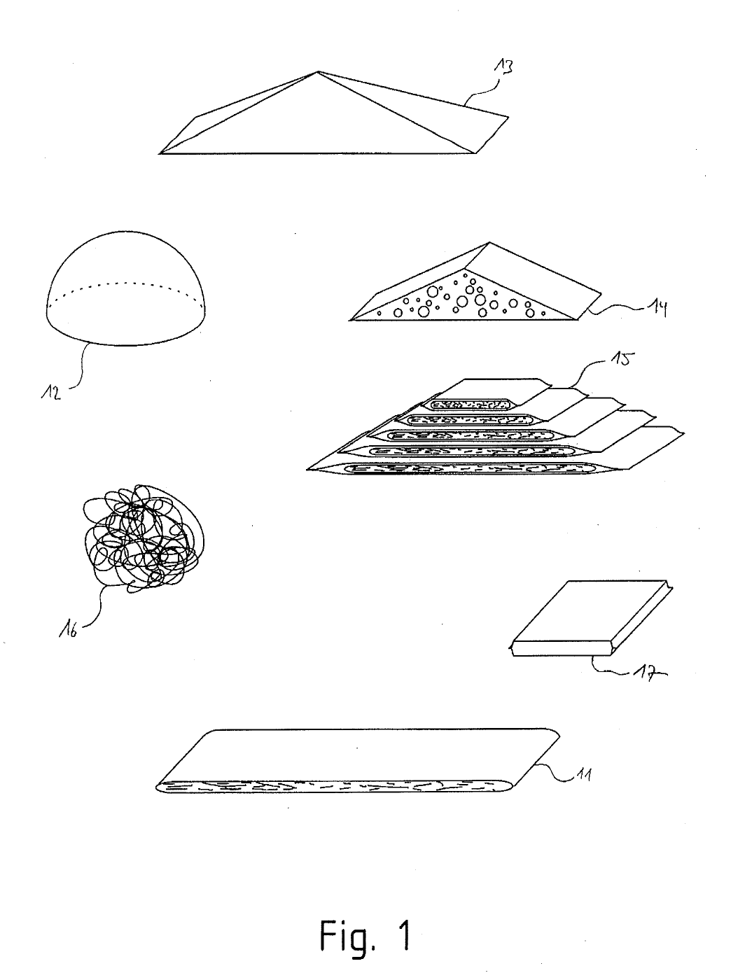

FIG. 1 shows different possible components of such an inventive wound care article. In the process first a flat layer 11 is provided, containing an absorbent material, in addition however at least one active element which gives the wound care article a convex or pyramidal shape when viewed from the side.

In this connection it can be e.g. a hemispherical active element 2, a pyramidal active element 13, a prismatic active element 14, a wad-like active element 16 or a cushion-like active element 17, preferably containing microspheres. Not shown, but nevertheless covered by the disclosure content of the present invention, are cone-shaped, bullet-shaped, tetrahedral, prismoid semispherical, spherical segment-shaped and similar shaped active elements.

Likewise said active element can be a multi-layer arrangement of individually wrapped flat layers 15 differing in size, containing an absorbent material.

Especially preferably provision can be made that the relevant active elements are equipped as deposits and contain specific active ingredients (for example a nutritive active ingredient, a disinfectant or decontaminating active ingredient and/or a protease inhibiting active ingredient and/or active ingredient complex) or superabsorbent polymers.

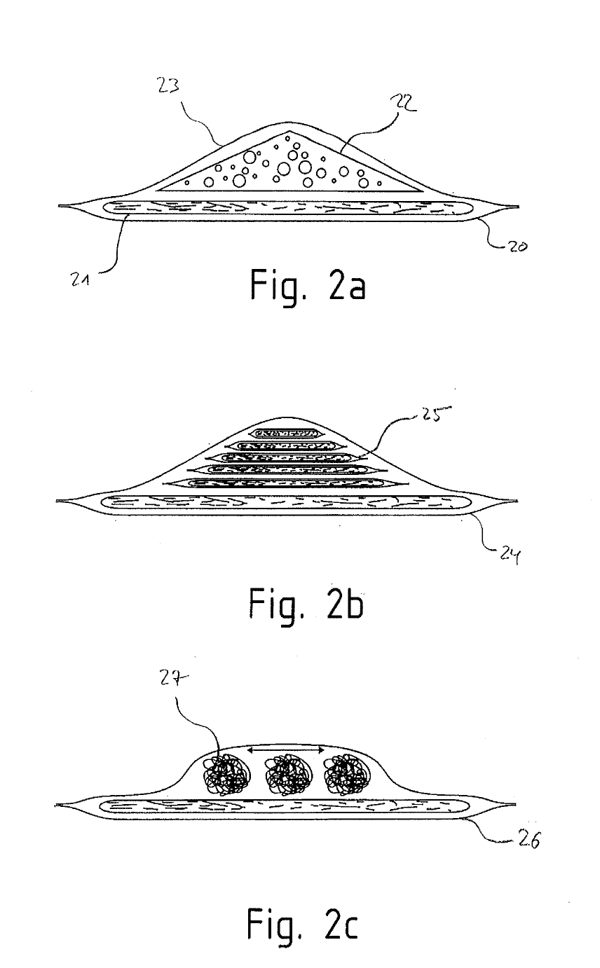

FIG. 2a shows such an inventive wound care article 20 exhibiting a flat layer 21, containing an absorbent material, an active element which gives the wound care article a convex or pyramidal shape when viewed from the side in the form of a prismatic log 22, which consists of foam, as well as a sleeve 23 consisting of 2 lengths which wraps the entire wound care article. The said sleeve consists preferably of a non-woven, e.g. a polypropylene non-woven, and is welded together on its edges via ultrasound seams.

FIG. 2b shows a similar wound care article 24, in which case the at least one active element, which gives the wound care article a convex or pyramidal shape, consists of a multi-layer arrangement 25 of flat, individually wrapped layers varying in size, containing an absorbent material.

FIG. 2c shows a further wound care article 26, in which case several wad-like active elements 27 are provided. These consist for example of cellulose wadding or a mixture of cellulose fibers and superabsorbent fibers. They can in particular be adjustably arranged in order to also adapt the convex shape of the wound care article to the wound to be treated. In deviation from FIG. 2c the wad-like active elements can also be provided with a separate wrapping.

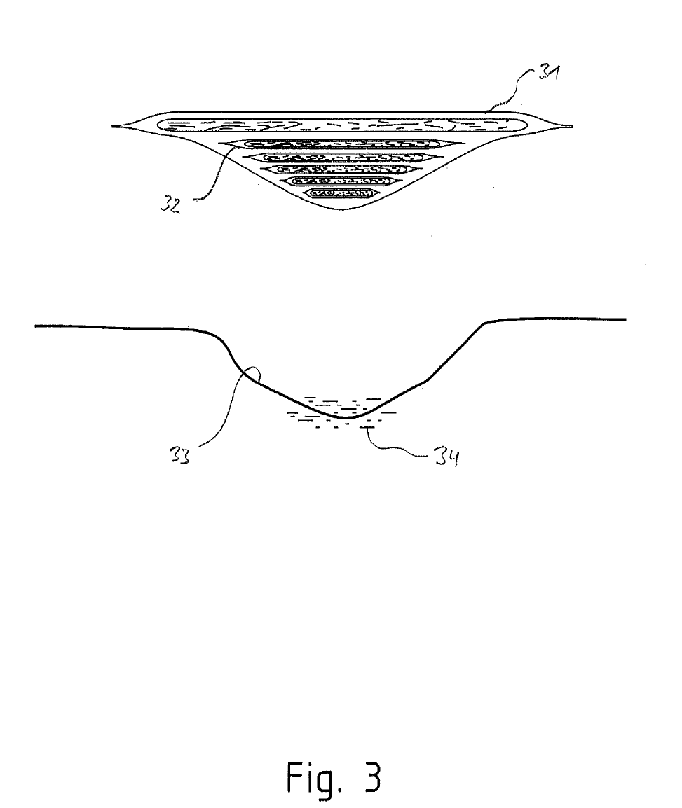

FIG. 3 shows an inventive wound care article 31 with an active element 32 which gives the wound care article a convex or pyramidal shape when viewed from the side on its planar arrangement as well as its arrangability in a deep wound 33. The wound care article can in this way fill deep wounds and come into contact with the base of the wound over wide areas. In this way deep edema and exudate accumulations are also covered, and the wound healing process is promoted.

Through the named convex shape in addition the superabsorbent polymers are brought quite close to the active site—in contrast to the above described wound fillers, which as a rule are passive, that is, not equipped with active agents.

This is particularly advantageous, since superabsorbent polymers can bind proteins and bacteria, as e.g. shown in DE102007054127 from the applicant of the present invention. Therefore the closer they are brought to the wound, the more advantageous the effect this property has on the healing of wounds.

1. A wound care article exhibiting at least one flat layer containing an absorbent material, wherein the wound care article exhibits at least one further active element which gives the wound care article a convex or pyramidal shape when viewed from the side.

2. The wound dressing according to claim 1, wherein the aforementioned at least one active element is a pyramidal, prismatic or hemispherical active element made of a soft and/or elastic material.

3. The wound dressing according to claim 1 wherein said soft and/or elastic material is selected from the group containing foam, silicone, hydrocolloid material, mull, gauze, fluff and/or dental impression material.

4. The wound dressing according to claim 1, wherein the at least one active element it is a matter of a multi-layer arrangement of flat, individually wrapped layers varying in size.

5. The wound dressing according to claim 1, wherein at least one of the said layers exhibits a segment made of fleece, preferably one made of airlaid.

6. The wound dressing according to claim 1, wherein the wound care article contains superabsorbent polymers at least in sections.

7. The wound dressing according to claim 1, wherein the wound care article exhibits in addition a liquid permeable sleeve.

8. The use of a wound care article according to claim 1, wherein for the treatment of deep wounds.

9. The wound dressing according to claim 2, wherein said soft and/or elastic material is selected from the group containing foam, silicone, hydrocolloid material, mull, gauze, fluff and/or dental impression material.
